(19) United States
(12) Patent Application Publication
(10) Pub. No.: US 2023/0372402 A1
(43) Pub. Date: Nov. 23, 2023

(54) CYTOKINE PRIMED REGENERATIVE CELLS FOR TREATMENT OF OVARIAN FAILURE

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/311,625

(22) Filed: May 3, 2023

Related U.S. Application Data
(60) Provisional application No. 63/343,832, filed on May 19, 2022.

(51) Int. Cl. A61K 35/28; A61K 38/18; C12N 5/0775

(52) U.S. Cl. CPC A61K 35/28; A61K 38/18; C12N 5/0662; C12N 2501/12; C12N 2501/115; C12N 2501/15; C12N 2501/105; C12N 2501/734

(57) ABSTRACT

Disclosed are novel means of generating cells uniquely suited for treatment of ovarian failure. In one embodiment regenerative cells are pretreated with growth factor-comprising composition(s), wherein the growth factor(s) may be cytokines, peptides, and/or proteins. In another embodiment regenerative cells are cultured with primed plasma extracts. In another embodiment, regenerative cells are cultured under hypoxic conditions together with cytokines prior to administration to an individual. Regenerative cells useful for the current invention including mesenchymal and hematopoietic stem cells, as well as various growth factor producing cells.

### CROSS-REFERENCE TO RELATED APPLICATIONS

This application claims priority to U.S. Provisional Application No. 63/343,832, titled “Cytokine Primed Regenerative Cells for Treatment of Ovarian Failure” filed May 19, 2022, which is hereby incorporated by reference herein in its entirety.

### FIELD OF THE INVENTION

The invention relates to the use of stem cell therapy to regenerate damaged ovaries in patients in need.

### BACKGROUND

Ovarian failure involves loss of functional female reproductive tissue. Premature ovarian failure is a clinical condition which occurs while ovarian follicles in the ovary disappear or do not respond due to various factors. Premature ovarian failure occurs in about 1% of women before the age of 40, and it is known that 10 to 28% of primary amenorrhea and 4 to 18% of secondary amenorrhea occur due to premature ovarian failure. Unlike menopause where the ovary completely ceases to function, pregnancy may be possible in 5 to 10% of premature ovarian failure, but the prognosis of premature ovarian failure cannot be inferred. Such premature ovarian failure may be caused by genetic factors and ovarian damage due to autoimmunity, and examples of the causes of premature ovarian failure include iatrogenic causes such as anti-cancer treatment, radiation treatment, and ovarian surgery, or virus infection, or environmental factors such as drugs and smoking, but there are many cases where it is difficult to explain the occurrence mechanism and causes of premature ovarian failure in most patients. For such premature ovarian failure, early menopause may cause patients to complain of more serious menopause-related symptoms earlier and more obviously than natural menopausal women. Early menopause may become a major antecedent cause of not only impairment of emotional well-being and sexual self, but also deterioration in health-related quality of life such as cardiovascular diseases and osteoporosis, in consideration of life expectancy after menopause.

To date there are no consistent method of treatment menopause or premature ovarian failures using current techniques.

### Summary

Preferred embodiments are directed to methods of augmenting efficacy of regenerative cells for repair of ovarian cells and/or tissues, comprising the steps of contacting regenerative cells with one or more biologically active substances; and/or incubating the regenerative cells under conditions to enhance efficacy of said regenerative cells for ovarian repair.

Preferred methods are directed to embodiments wherein said biologically active substance comprises one or more cytokines.

Preferred methods are directed to embodiments wherein said cytokine comprises one or more growth factors.

Preferred methods are directed to embodiments wherein said growth factor is FGF-alpha.

Preferred methods are directed to embodiments wherein said growth factor is FGF-beta.

Preferred methods are directed to embodiments wherein said growth factor is interleukin-10.

Preferred methods are directed to embodiments wherein said growth factor is amphiregulin.

Preferred methods are directed to embodiments wherein said growth factor is endoglin.

Preferred methods are directed to embodiments wherein said growth factor is PDGF-BB.

Preferred methods are directed to embodiments wherein said growth factor is IGF.

Preferred methods are directed to embodiments wherein said growth factor is CTGF.

Preferred methods are directed to embodiments wherein said growth factor is HGF.

Preferred methods are directed to embodiments wherein said growth factor is FGF-beta.

Preferred methods are directed to embodiments wherein said growth factor is a member of the TGF-beta family.

Preferred methods are directed to embodiments wherein said biologically active substance comprises platelet rich plasma.

Preferred methods are directed to embodiments wherein said regeneration comprises immune modulation.

Preferred methods are directed to embodiments wherein said regeneration comprises angiogenesis.

Preferred methods are directed to embodiments wherein said regeneration is regeneration of spinal discs.

Preferred methods are directed to embodiments wherein the fibroblast cells are selected from the group consisting of (a) regenerative cell obtained by biopsy, cultured and proliferated; (b) subsets thereof having greater ability to differentiate; and (c) a combination thereof.

Preferred methods are directed to embodiments wherein the regenerative cells express stage specific embryonic antigen 3 (SSEA3).

Preferred methods are directed to embodiments wherein said regenerative cells are comprised in a pharmaceutically acceptable carrier selected from the group consisting of sterile solutions, hydrogels, implantable cell matrices, devices and a combination thereof.

Preferred methods are directed to embodiments wherein the regenerative cells are derived from tissues comprising skin, heart, blood vessels, bone marrow, skeletal muscle, liver, pancreas, brain, adipose tissue, foreskin, placental, and/or umbilical cord.

Preferred methods are directed to embodiments wherein the regenerative cells are placental, fetal, neonatal, adult or a combination thereof.

Preferred methods are directed to embodiments wherein said biologically active substance is a protease.

Preferred methods are directed to embodiments wherein said protease is a collagenase.

Preferred methods are directed to embodiments wherein said protease is a hydroxylase.

Preferred methods are directed to embodiments wherein said biologically active substance is a matrix metalloprotease.

Preferred methods are directed to embodiments wherein said matrix metalloprotease is MMP1.

Preferred methods are directed to embodiments wherein said matrix metalloprotease is MMP2.

Preferred methods are directed to embodiments wherein said matrix metalloprotease is MMP3.

Preferred methods are directed to embodiments wherein said matrix metalloprotease is MMP7.

Preferred methods are directed to embodiments wherein said matrix metalloprotease is MMP9.

Preferred methods are directed to embodiments wherein said matrix metalloprotease is MMP13.

Preferred methods are directed to embodiments wherein said cell population is autologous.

Preferred methods are directed to embodiments wherein said cell population is allogeneic.

Preferred methods are directed to embodiments wherein said cell population is xenogenic.

Preferred methods are directed to embodiments wherein said cell population is bone marrow mononuclear cells.

Preferred methods are directed to embodiments wherein said cell population is mesenchymal stem cells.

Preferred methods are directed to embodiments wherein said cell population is amniotic stem cells.

Preferred methods are directed to embodiments wherein said cell population is embryonic stem cells.

Preferred methods are directed to embodiments wherein said cell population is inducible pluripotent stem cells.

Preferred methods are directed to embodiments wherein said cell population is a hematopoietic stem cell population.

Preferred methods are directed to embodiments wherein said mesenchymal stem cells express CD90.

Preferred methods are directed to embodiments wherein said mesenchymal stem cells express CD105.

Preferred methods are directed to embodiments wherein said mesenchymal stem cells express c-met.

Preferred methods are directed to embodiments wherein said mesenchymal stem cells express CD133.

Preferred methods are directed to embodiments wherein said mesenchymal stem cells express c-kit.

Preferred methods are directed to embodiments wherein said mesenchymal stem cells express IL-1 receptor.

Preferred methods are directed to embodiments wherein said mesenchymal stem cells express IL-1 receptor antagonist when treated with interferon gamma.

Preferred methods are directed to embodiments wherein said interferon gamma is administered at a concentration of 5 ng/ml for a period of 1 hour to 24 hours.

Preferred methods are directed to embodiments wherein said regenerative cells are administered together with a growth factor.

Preferred methods are directed to embodiments wherein said growth factor is hepatocyte growth factor.

Preferred methods are directed to embodiments wherein said growth factor is epidermal growth factor.

Preferred methods are directed to embodiments wherein said growth factor is insulin growth factor.

Preferred methods are directed to embodiments wherein said growth factor is keratinocyte growth factor.

Preferred methods are directed to embodiments wherein said growth factor is PDGF-BB.

### DETAILED DESCRIPTION OF THE INVENTION

The present disclosure is directed to methods and compositions related to certain cells useful for therapy in an individual, such as therapy in a female suffering from ovarian failure, or having susceptibility to ovarian. In specific embodiments, the cells are regenerative cells, in some cases, stem cells, in particular cases hematopoietic or mesenchymal stem cells. In particular cases, said cells have been modified upon exposure to one or more biologically active substance and/or one or more conditions. In certain embodiments the exposure improves one or more therapeutic activities compared to regenerative cells that lack the exposure. Although any particular therapeutic activity of the regenerative cells may be enhanced upon one or more exposures to one or more biologically active substance and/or one or more conditions, in some cases the activity is anti-inflammatory, angiogenic, regenerative and/or ovarian-regenerating properties, as examples. In specific cases wherein the fibroblasts have improved activities, the fibroblasts are directly or indirectly the cause of amelioration of at least one symptom of a medical condition related to ovarian failure of an individual.

In particular aspects of the invention, methods of the disclosure directly or indirectly result in an increase ovarian volume, regeneration of oocyte, and granulosa cells, including by increasing synthesis in the ovary, by decreasing degradation, and/or by preventing matrix loss by inhibiting degradative enzymes. In one embodiment, methods are provided for augmenting efficacy of stem cells for regeneration of cells and/or tissues, comprising the steps of (optionally) obtaining stem cells; contacting stem cells with one or more biologically active substances; and/or culturing the stem cells under conditions to enhance efficacy of the stem cells for regeneration of the cells and/or tissues. In specific embodiments, the one or more biologically active substances comprise one or more cytokines, such as growth factors (for example, FGF-alpha, FGF-beta, and/or a member of the TGF-beta family). In specific cases, the one or more biologically active substances comprise platelet rich plasma. In particular embodiments, regeneration of cells and/or tissue by the stem cells comprises immune modulation, angiogenesis, regeneration of ovarian tissue and/or function, a combination thereof, and so forth. The stem cells being utilized may be selected from the group consisting of (a) stem cells obtained by biopsy, cultured and proliferated; (b) subsets thereof having greater ability to differentiate; and (c) a combination thereof. In specific cases, the stem cells express stage specific embryonic antigen 3 (SSEA3). In certain cases, the stem cells are comprised in a pharmaceutically acceptable carrier selected from the group consisting of sterile solutions, hydrogels, implantable cell matrices, devices and a combination thereof. Some aspects of the invention disclosed methods for increasing estrogen production and expression in stem cells, suppressing T-cell activation by stem cells, and/or suppressing T-cell production of one or more factors, such as interferon gamma, by stem cells.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

“Biocompatible polymers” used in the present disclosure are selected from the group consisting of carbomers (acrylic acid polymers crosslinked with a polyalkenyl polyether), polyalkylene glycols (for example, polyethylene glycols and polypropylene glycols), poloxamers (polyoxyethylene-polyoxypropylene block copolymers), polyesters, polyethers, polyanhydrides, polyacrylates, polyvinyl acetates, polyvinyl pyrrolidones, and polysaccharides such as, for example, hyaluronic acid, derivatives of hyaluronic acid, in particular crosslinked hyaluronic acid and esters of hyaluronic acid (for example, benzyl ester of hyaluronic acid), hydroxyalkylcelluloses (for example, hydroxymethylcellulose and hydroxyethylcellulose), and carboxyalkylcelluloses (for example, carboxymethylcellulose).

“Growth factor” refers to any material or materials having a positive reaction on living tissues, such as promoting the growth of tissues. Exemplary growth factors include, but are not limited to, platelet-derived growth factor (PDGF), platelet-derived angiogenesis factor (PDAF), vascular endothelial growth factor (VEGF), platelet-derived epidermal growth factor (PDEGF), platelet factor 4 (PF-4), transforming growth factor beta. (TGF-B), acidic fibroblast growth factor (FGF-A), basic fibroblast growth factor (FGF-B), transforming growth factor A (TGF-A), insulin-like growth factors 1 and 2 (IGF-1 and IGF-2), B thromboglobulin-related proteins (BTG), thrombospondin (TSP), fibronectin, von Wallinbrand's factor (vWF), fibropeptide A, fibrinogen, albumin, plasminogen activator inhibitor 1 (PAI-1), osteonectin, regulated upon activation normal T cell expressed and presumably secreted (RANTES), gro-A, vitronectin, fibrin D-dimer, factor V, antithrombin III, immunoglobulin-G (IgG), immunoglobulin-M (IgM), immunoglobulin-A (IgA), a2-macroglobulin, angiogenin, Fg-D, elastase, keratinocyte growth factor (KGF), epidermal growth factor (EGF), fibroblast growth factor (FGF), tumor necrosis factor (TNF), fibroblast growth factor (FGF) and interleukin-1 (IL-1), Keratinocyte Growth Factor-2 (KGF-2), and combinations thereof. One of the important characteristics common to the above listed growth factors is that each substance is known or believed to have a positive reaction on living tissue, known as bioactivity, to enhance cell or tissue growth.

The term “mesenchymal stem cell” refers to but in no way is limited to, those described in the following references, the disclosures of which are incorporated herein by reference: U.S. Pat. Nos. 5,215,927; 5,225,353; 5,262,334; 5,240,856; 5,486,359; 5,759,793; 5,827,735; 5,811,094; 5,736,396; 5,837,539; 5,837,670; 5,827,740; 6,087,113; 6,387,367; 7,060,494; 8,790,638; Jaiswal, N., et al., J. Cell Biochem. (1997) 64(2): 295 312; Cassiede P., et al., J. Bone Miner. Res. (1996) 11(9): 1264 1273; Johnstone, B., et al., (1998) 238(1): 265 272; Yoo, et al., J. Bone Joint Sure. Am. (1998) 80(12): 1745 1757; Gronthos, S., Blood (1994) 84(12): 41644173; Basch, et al., J. Immunol. Methods (1983) 56: 269; Wysocki and Sato, Proc. Natl. Acad. Sci. (USA) (1978) 75: 2844; and Makino, S., et al., J. Clin. Invest. (1999) 103(5): 697 705.

The term “regeneration” as used herein refers to the growth of new cells and/or tissue in an area, such as a damaged area, including ovarian tissue.

The disclosure concerns means of augmenting therapeutic activity of regenerative cells for the treatment of ovarian failure, such as regenerative cells that are used at least for anti-inflammatory, angiogenic, regenerative and/or ovary-regenerating properties at one or more sites in vivo. In one embodiment of the disclosure, regenerative cells are cultured with cytokines, growth factors, peptides, or combinations thereof prior to administration to an individual, such as a mammal, including humans, horses, dogs, cats, and so forth. In another embodiment, the disclosure encompasses augmentation of regenerative activities for stem cells to be used as therapeutic agents, for example through culture (before and/or during administration to an individual) with one or more agents, such as platelet rich plasma (PRP). In another embodiment the disclosure provides methods for enhancing one or more fibroblast activities for therapeutic activity by co-administering one or more agents and/or PRP, for example together with the regenerative cells such as stem cells. In particular cases the enhanced fibroblasts are delivered to an individual for the purpose of treating a ovarian medical condition in the individual. In some cases an individual is determined to be in need of the enhanced stem cell activity, such as because of ovarian failure or risk thereof. An individual at risk is one that is over the age of about 40, 45, 50, 55, 60, 65, 70, 75, 80, and so forth. In some embodiments, the regenerative cells are exposed to platelet-rich plasma and such exposure directly or indirectly results in enhanced regenerative cells. Numerous growth factors, cytokines and peptides are released from activated platelets, and one approach to therapeutically leverage this is to utilize an autologous platelet concentrate suspended in plasma, also known as platelet-rich plasma (PRP). Several means of preparing PRP are known in the art, some of which are described in the following and incorporated by reference herein [36, 37]. Examples of devices used for generation of PRP include SmartPReP, 3iPCCS, Sequestra, Secquire, CATS, Interpore Cross, Biomet GPS, Cervos and Harvest's BMAC [38], for example. Other means of generating PRP are described in U.S. Pat. Nos. 5,585,007; 5,599,558; 5,614,204; 6,214,338; 6,010,627; 5,165,928; 6,303,112; 6,649,072; and 6,649,072, which are incorporated by reference herein in their entirety. In specific embodiments, one can dose PRP at the time of injection in the individual, such as without a prior culture with the fibroblasts. In one embodiment of the disclosure, regenerative cells are delivered systemically or locally to an individual in need thereof, including an individual in need of treatment, including by using a carrier (for example, hydrogel) comprising platelet rich plasma (PRP) and/or hyaluronic acid (HA); in particular cases PRP and/or HA are blended with batroxobin (BTX) as gelling agent. The regenerative cells may be encapsulated in a hydrogel, such as PRP/HA/BTX hydrogel, and cultured, for example in both growing medium and/or medium with or without TGF-01 (for example) for a certain duration of time, such as from one minute (min) up to 21 days. A range of culture duration for any cells may be from 1, 2, 3, 4, 5, 6, 7, 8, 9, 10, 15, 20, 25, 30, 35, 40, 45, 50, 55, 60 (or more minutes or from 1, 2, 3, 4, 5, 6, 7, 8, 9, 10, 11, 12, 13, 14, 15, 16, 17, 18, 19, 20, 21, 22, 23, or 24 or more hours) to 1, 2, 3, 4, 5, 6, 7, 8, 9, 10, 11, 12, 13, 14, 15, 16, 17, 18, 19, 20, or 21 days. The range of time may be from 1, 2, 3, 4, 5, 6, 7, 8, 9, 10, 15, 20, 25, 30, 35, 40, 45, 50, 55, 60 or more minutes to 1, 2, 3, 4, 5, 6, 7, 8, 9, 10, 11, 12, 13, 14, 15, 16, 17, 18, 19, 20, 21, 22, 23, or 24 or more hours. In one embodiment, intra-ovarian administration of stem cells and the hydrogel is performed, which results in jellifies at a certain temperature in a certain period of time. The hydrogel may jellify in 10, 11, 12, 13, 14, 15, 16, 17, 18, 19, or 20 minutes or more at 18, 19, 20, 21, 22, 23, 24, 25, or higher .degree. C. or in 1, 2, 3, 4, 5, or more minutes at 35, 36, 37, 38, 39, or 40 or more .degree. C. in a manner such that the regenerative cells maintain high cell viability and proliferation. In one embodiment the disclosure encompasses the use of fibroblasts for local delivery (such as by intra-disc injections) in individuals with degenerative ovarian failure. In such an embodiment, the regenerative cells are cultured in suitable conditions to enhance GAG production, which in at least some cases is achieved by culture with one or more cytokines, such as TGF-beta. Methodologies for growth of mesenchymal stem cells, is incorporated by reference [39].

The disclosure encompasses the use of activation of regenerative cells prior to therapeutic use, including administration of one or more biologically active substances that act as “regenerative adjuvants” for the fibroblasts. The cells in the formulation may display typical regenerative cells morphologies when growing in cultured monolayers. Specifically, cells may display an elongated, fusiform or spindle appearance with slender extensions, or cells may appear as larger, flattened stellate cells that may have cytoplasmic leading edges. A mixture of these morphologies may also be observed. The cells may express one or more proteins characteristic of normal regenerative cells including the fibroblast-specific marker, CD90 (Thy-1), a 35 kDa cell-surface glycoprotein, and the extracellular matrix protein, collagen, as examples. The fibroblast dosage formulation in specific embodiments may be an autologous, allogeneic, or xenogeneic cell therapy product comprising a suspension of regenerative cells, including grown from skin using standard tissue culture procedures as examples.

In certain embodiments, regenerative cells of any kind are utilized in methods for regeneration, and in preparation for (or as part of) these methods, the fibroblasts may be harvested, cultured, and expanded using certain techniques.

Following the obtaining and preparation of regenerative cells prior to delivery to an individual in need thereof, the regenerative cells may be manipulated, including to enhance one or more activities useful for a therapeutic purpose. In some cases, the regenerative cells are exposed to one or more biologically active agents and/or conditions prior to (and/or during) delivery to an individual in need thereof, and in some cases the exposure to one or more biologically active agents and/or conditions prior to (and/or during) delivery may or may not occur during a culturing step. In one embodiment, regenerative cells are pre-activated by contact with a composition or mixture of compositions comprising a biologically active agent that is at least one growth factor, and the growth factor(s) may be selected from the group consisting of transforming growth factors (TGF), fibroblast growth factors (FGF), platelet-derived growth factors (PDGF), epidermal growth factors (EGF), vascular endothelial growth factors (VEGF), insulin-like growth factors (IGF), platelet-derived endothelial growth factors (PDEGF), platelet-derived angiogenesis factors (PDAF), platelet factors 4 (PF-4), hepatocyte growth factors (HGF) and a combination thereof. In certain cases, the growth factors are transforming growth factors (TGF), platelet-derived growth factors (PDGF) fibroblast growth factors (FGF) and a combination thereof. In specific cases, the growth factors are selected from the group consisting of transforming growth factors beta (TGF-beta), platelet-derived growth factors BB (PDGF-BB), basic fibroblast growth factors (bFGF) and a combination thereof. In another embodiment of the disclosure, the growth factor-comprising compositions are delivered to an individual simultaneously with, or subsequent to, delivery of regenerative cells. The delivery may occur by injection, in certain embodiments. The regenerative cells may be autologous, allogeneic, or xenogeneic with respect to the recipient individual. In some embodiments a platelet plasma composition is administered together with the regenerative cells or subsequent to administration of the fibroblasts, and the platelet plasma composition may comprise, consist essentially of, or consist of platelets and plasma and may be derived from bone marrow and/or peripheral blood. The present disclosure may use platelet plasma composition(s) from either or both of these sources, and either platelet plasma composition may be used to regenerate either a nucleus or annulus or both in need thereof. Further, the platelet plasma composition may be used with or without concentrated bone marrow (BMAC). By way of example, when inserted into the annulus, 0.05-2.0 cc of platelet plasma composition may be used, and when inserted into the nucleus, 0.05-3.0 cc of the platelet plasma composition may be used. Platelets are non-nucleated blood cells that as noted above may be found in bone marrow and peripheral blood. In various embodiments of the present disclosure, a platelet plasma composition may be obtained by sequestering platelets from whole blood and/or bone marrow through centrifugation, for example into three strata: (1) platelet rich plasma; (2) platelet poor plasma; and (3) fibrinogen. When using platelets from one of the strata, e.g., the platelet rich plasma (PRP) from blood, one may use the platelets whole or their contents may be extracted and concentrated into a platelet lysate through a cell membrane lysis procedure using thrombin and/or calcium chloride, for example. When choosing whether to use the platelets whole or as a lysate, one may consider the rate at which one desires regeneration and/or tissue healing (which may include the formation of scar tissue without regeneration or healing of a herniated or torn disc). In some embodiments the lysate will act more rapidly than the PRP (or platelet poor plasma from bone marrow). Notably, platelet poor plasma that is derived from bone marrow has a greater platelet concentration than platelet rich plasma from blood, also known as platelet poor/rich plasma, (“PP/RP” or “PPP”). PP/RP or PPP may be used to refer to platelet poor plasma derived from bone marrow, and in some embodiments, preferably PP/RP is used or PRP is used as part of the composition for ovary regeneration. (By convention, the abbreviation PRP refers only to compositions derived from peripheral blood and PPP (or PP/RP) refers to compositions derived from bone marrow

In some embodiments in which the lysate is used, the cytokine(s) may be concentrated in order to optimize their functional capacity. Concentration may be accomplished in two steps. First, blood may be obtained and concentrated to a volume that is 5-15% of what it was before concentration. Devices that may be used include but are not limited to a hemofilter or a hemoconcentrator. For example, 60 cc of blood may be concentrated down to 6 cc. Next, the concentrated blood may be filtered to remove water. This filtering step may reduce the volume further to 33%-67% (e.g., approximately 50%) of what it was prior to filtration. Thus, by way of example for a concentration product of 6 cc, one may filter out water so that one obtains a product of approximately 3 cc. When the platelet rich plasma, platelet poor plasma and fibrinogen are obtained from blood, they may for example be obtained by drawing 20-500 cc of peripheral blood, 40-250 cc of peripheral blood or 60-100 cc of peripheral blood. The amount of blood that one should draw will depend on the number of discs that have degenerated and the size of the discs. As persons of ordinary skill in the art will appreciate, a typical disc has a volume of 2-5 cc or 3-4 cc. In one specific embodiment regenerative cells are treated, or administered together with activated PRP. The method of generation of activated PRP may be used according to U.S. Pat. No. 9,011,929, which is incorporated by reference herein in its entirety and describes essentially: separating the PRP from whole blood, wherein the separating step further comprises the steps of: collecting 10 ml of the whole blood from an animal or patient into a vacuum test tube containing 3.2% sodium citrate, and primarily centrifuging the collected whole blood at 1,750-1,900 g for 3 to 5 minutes; collecting a supernatant liquid comprising a plasma layer with a buffy coat obtained from said centrifugation; transferring the collected supernatant liquid to a new vacuum test tube by a blunt needle, and secondarily centrifuging the collected supernatant liquid at 4,500-5,000 g for 4 to 6 minutes; and collecting the PRP concentrated in a bottom layer by another blunt needle; mixing 1 mL of the PRP collected from the separating step with a calcium chloride solution with a concentration of 0.30-0.55 mg/mL by a three-way connector; and mixing a mixture of the PRP and the calcium chloride solution with type I collagen, wherein the mixing step of mixing the mixture of the PRP and the calcium chloride solution with the type I collagen further comprises the steps of: leaving the type I collagen at a room temperature for 15 to 30 minutes before mixing; and mixing the mixture of the PRP and the calcium chloride solution with the type I collagen with a concentration of 20-50 mg/mL, in an opaque phase, four times by another three-way connector. In some embodiments administration of fibroblasts is performed together with biocompatible polymers and growth factors or PRP, or Platelet Gel.

Treatment of individuals with ovarian failure may be accomplished through one embodiment of the disclosure, such as through the administration of regenerative cells that have been genetically modified to upregulate expression of angiogenic stimuli or anti-inflammatory activities. It is known in the art that genes may be introduced by a wide range of approaches including adenoviral, adeno-associated, retroviral, alpha-viral, lentiviral, Kunjin virus, or HSV vectors, liposomal, nano-particle mediated as well as electroporation and Sleeping Beauty transposons. Genes with angiogenic stimulatory function that may be transfected include but are not limited to: VEGF, FGF-1, FGF-2, FGF-4, EGF, HGF, and a combination thereof. Additionally, transcription factors that are associated with upregulating expression of angiogenic cascades may also be transfected into cells used for treatment of lower back pain, including: HIF-1alpha, HIF-2, NET, NF-kB, or a combination thereof. Genes inhibitory to inflammation may be used such as: TGF-a, TGF-b, IL-4, IL-10, IL-13, IL-20 thrombospondin, or a combination thereof, for example. Transfection may also be utilized for administration of genetic manipulation means in a manner to substantially block transcription or translation of genes which inhibit angiogenesis. Antisense oligonucleotides, ribozymes or short interfering RNA may be transfected into cells for use for treatment of lower back pain in order to block expression of antiangiogenic proteins such as: canstatin, IP-10, kringle 1-5, and collagen XVIII/endostatin, for example. Additionally, gene inhibitory technologies may be used for blocking ability of cells to be used for treatment of lower back pain to express inflammatory proteins including: IL-1, TNF-alpha, IL-2, IL-6, IL-8, IL-9, IL-11, IL-12, IL-15, IL-17, IL-18, IL-21, IL-23, IL-27, IFN-alpha, IFN-beta, and IFN-gamma. Globally acting transcription factors associated with inflammation may also be substantially blocked using not only the genetic means described but also decoy oligonucleotides. Suitable transcription factors for blocking include various subunits of the NF-kB complex such as p55, and/or p60, STAT family members, particularly STAT1, STATS, STAT4, and members of the Interferon Regulatory Factor family such as IRF-1, IFR-3, and IFR-8, for example. Enhancement of angiogenic stimulation ability of the cells useful for the treatment of back pain can be performed through culturing under conditions of restricted oxygen. It is known in the art that stem cells in general, and ones with angiogenesis promoting activity specifically, tend to reside in hypoxia niches of the bone marrow. When stem cells differentiate into more mature progeny, they progressively migrate to areas of the bone marrow with higher oxygen tension.[40]. This important variable in tissue culture was used in studies that demonstrated superior expansion of human CD34 stem cells capable of full hematopoietic reconstitution were obtained in hypoxic conditions using oxygen tension as low as 1.5%. The potent expansion under hypoxia, which was 5.8-fold higher as compared to normal oxygen tension was attributed to hypoxia induction of HIF-1 dependent growth factors such as VEGF, which are potent angiogenic stimuli when released under controlled conditions [41]. Accordingly, culture of cells to be used for treatment of back pain may be performed in conditions of oxygen ranging from 0.5% to 4%, such as 1%-3% and including from 1.5%-1.9%. Hypoxia culture is not limited towards lowering oxygen tension but may also include administration of molecules that inhibit oxygen uptake or compete with oxygen uptake during the tissue culture process. Additionally, in an embodiment of the disclosure, hypoxia is induced through induction of one or more agents that cause the upregulation of the HIF-1 transcription factor. In embodiments wherein the regenerative cells are exposed to hypoxia, the oxygen levels may be between 0.1%-5%, 0.1%-4%, 0.1%-3%, 0.1%-2%, 0.1%-1%, 0.1%-0.75%, 0.1%-0.5%, 0.1%-0.25%, 0.2%-5%, 0.2%-4%, 0.2%-3%, 0.2%-2%, 0.2%-1%, 0.2%-0.75%, 0.2%-0.5%, 0.5%-5%, 0.5%-4%, 0.5%-3%, 0.5%-2%, 0.5%-1%, 0.5%-0.75%, 0.75%-5%, 0.75%-4%, 0.75%-3%, 0.75%-2%, 0.75%-1%, 1%-5%, 1%-4%, 1%-3%, 1%-2%, 2%-5%, 2%-4%, 2%-3%, 3%-5%, 3%-4%, or 4%-5%% oxygen, in specific embodiments. The duration of exposure of the cells to hypoxic conditions, including with (but not limited to) these representative levels of oxygen, may be for a duration of 30 minutes (min)-3 days, 30 min-2 days, 30 min-1 day, 30 min-12 hours (hrs), 30 min-8 hrs, 30 min-6 hrs, 30 min-4 hrs, 30 min-2 hrs, 30 min-1 hour (hr), 1 hr-3 days, 1 hr-2 days, 1 hr-1 day, 1-12 hrs, 1-8 hrs, 1-6 hrs, 1-4 hrs, 1-2 hrs, 2 hrs-3 days, 2 hrs-2 days, 2 hrs-1 day, 2 hrs-12 hrs, 2-10 hrs, 2-8 hrs, 2-6 hrs, 2-4 hrs, 2-3 hrs, 6 hrs-3 days, 6 hrs-2 days, 6 hrs-1 day, 6-12 hrs, 6-8 hrs, 8 hrs-3 days, 8 hrs-2 days, 8 hrs-1 day, 8-16 hrs, 8-12 hrs, 8-10 hrs, 12 hrs-3 days, 12 hrs-2 days, 12 hrs-1 day, 12-18 hrs, 12-14 hrs, 1-3 days, or 1-2 days, as examples only.

Assessment of the anti-inflammatory abilities of regenerative cells generated or isolated for potential clinical use may also be performed. Numerous methods are known in the art, for example they may include assessment of the putative anti-inflammatory regenerative cells to modulate immunological parameters in vitro. Putative anti-inflammatory regenerative cells may be co-cultured at various ratios with an immunological cell. The immunological cell may be stimulated with an activatory stimulus. The ability of the putative anti-inflammatory cell to inhibit, in a dose-dependent manner, production of inflammatory cytokines or to augment production of anti-inflammatory cytokines, may be used as an output system of assessing anti-inflammatory activity. Additional output parameters may include: proliferation, cytotoxic activity, production of inflammatory mediators, or upregulation of surface markers associated with activation. Cytokines assessed may include: IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, TNF, IFN and/or RANKL. Specific immunological cells may be freshly isolated or may be immortalized cell lines. The immunological cells may be: T cells, B cells, monocytes, macrophages, neutrophils, eosinophils, basophils, dendritic cells, natural killer cells, natural killer T cells, gamma delta-T cells, or a combination thereof. The immunological stimuli may include an antibody, a ligand, a protein, or another cells. Examples including: crosslinking antibodies to T cell receptor, to costimulatory molecules such as CD28, to activation associated molecules such as CD69 or to receptors for stimulatory cytokines such as IL-2. Additional examples of inflammatory stimuli may include co-culture with allogeneic stimulator cells such as in mixed lymphocyte reactions, or may include stimulation with a non-specific activator such as a lectin. Specific lectins may include conconavalin-A, phytohemagluttinin, or wheat germ agglutinin. Other non-specific stimulators may be activators of the toll like receptor pathway such as lipopolysaccharide, CpG DNA motifs or bacterial membrane fractions. The methods described in the above two paragraphs are shown only as examples that may be used to determine, before entry into clinical use, whether a cell population generated as described in the present invention is capable of producing the desired angiogenic stimulatory or anti-inflammatory effects. These examples are only provided as guides which one skilled in the art can optimize upon using routine experimentation.

For any embodiments of the disclosure provided herein, cells to be used for treatment of ovarian failure may be cryopreserved for subsequent use, as well as for transportation, in some cases. One skilled in the art knows numerous methods of cellular cryopreservation. Typically, cells may be treated to a cryoprotection process, then stored frozen until needed. Once needed cells require specialized care for revival and washing to clear cryopreservative agents that may have detrimental effects on cellular function. Generally, cryopreservation requires attention be paid to three main concepts, these are: 1) the cryoprotective agent, 2) the control of the freezing rate, and 3) the temperature at which the cells will be stored. Cryoprotective agents are well known to one skilled in the art and can include but are not limited to dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose, or choline chloride as described in U.S. Pat. No. 6,461,645 (incorporated by reference herein in its entirety), for example. A method for cryopreservation of cells that is utilized by some skilled artisans comprises DMSO at a concentration not being immediately cytotoxic to cells under conditions which allow it to freely permeate the cell and to protect intracellular organelles; the DMSO combines with water and prevents cellular damage induced from ice crystal formation. Addition of plasma at concentrations between 20-25% by volume can augment the protective effect of DMSO. After addition of DMSO, cells should be kept at temperatures below 4 C, in order to prevent DMSO-mediated damage. Methods of actually inducing the cells in a state of suspended animation involve utilization of various cooling protocols. While cell type, freezing reagent, and concentration of cells are important variables in determining methods of cooling, it is generally accepted that a controlled, steady rate of cooling is optimal. There are numerous devices and apparatuses known in the field that are capable of reducing temperatures of cells for optimal cryopreservations. One such apparatus is the Thermo Electro Cryomed Freezer™ manufactured by Thermo Electron Corporation. Cells can also be frozen in CryoCyte™ containers as made by Baxter. One example of cryopreservation is as follows: 2.times.10.sup.6 CD34 cells/ml are isolated from cord blood using the Isolex System™ as per manufacturer's instructions (Baxter). Cells are incubated in DMEM media with 10% DMSO and 20% plasma. Cooling is performed at 1 Celsius./minute from 0 to -80 Celsius. When cells are needed for use, they are thawed rapidly in a water bath maintained at 37 Celsius water bath and chilled immediately upon thawing. Cells are rapidly washed, either a buffer solution, or a solution containing a growth factor. Purified cells can then be used for expansion if needed. A database of stored cell information (such as donor, cell origination types, cell markers, etc.) can also be prepared, if desired. In certain embodiments, regenerative cells may be derived from tissues comprising skin, heart, blood vessels, bone marrow, skeletal muscle, liver, pancreas, brain, adipose tissue, foreskin, placental, and/or umbilical cord, for example. In specific embodiments, the fibroblasts are placental, fetal, neonatal or adult or mixtures thereof. The number of administrations of cells to an individual will depend upon the factors described herein at least in part and may be optimized using routine methods in the art. In specific embodiments, a single administration is required. In other embodiments, a plurality of administration of cells is required. It should be appreciated that the system is subject to variables, such as the particular need of the individual, which may vary with time and circumstances, the rate of loss of the cellular activity as a result of loss of cells or activity of individual cells, and the like. Therefore, it is expected that each individual could be monitored for the proper dosage, and such practices of monitoring an individual are routine in the art.

In some embodiments, the cells are subjected to one or more media compositions that comprises, consists of, or consists essentially of Roswell Park Memorial Institute (RPMI-1640), Dublecco's Modified Essential Media (DMEM), Eagle's Modified Essential Media (EMEM), Optimem, Iscove's Media, or a combination thereof. In particular cases, the regenerative cells are recombinantly manipulated to encode SSEA3, VEGF, FGF-1, FGF-2, FGF-4, EGF, HGF, HIF-1alpha, HIF-2, NET, NF-kB, TGF-a, TGF-b, IL-4, IL-10, IL-13, IL-20 thrombospondin, canstatin, IP-10, kringle 1-5, collagen XVIII/endostatin, IL-1, TNF-alpha, IL-2, IL-6, IL-8, IL-9, IL-11, IL-12, IL-15, IL-17, IL-18, IL-21, IL-23, IL-27, IFN-alpha, IFN-beta, IFN-gamma, p55, p60, STAT1, STATS, STAT4, IRF-1, IFR-3, IFR-8, or a combination thereof. In cases wherein recombination technology is employed, one or more types of the fibroblast cells are manipulated to harbor one or more expression vectors that each encode one or more gene products of interest. A recombinant expression vector(s) can be introduced as one or more DNA molecules or constructs, where there may be at least one marker that will allow for selection of host cells that contain the vector(s). The vector(s) can be prepared in conventional ways, wherein the genes and regulatory regions may be isolated, as appropriate, ligated, cloned in an appropriate cloning host, and analyzed by sequencing or other convenient means. Particularly, using PCR, individual fragments including all or portions of a functional unit may be isolated, where in some cases one or more mutations may be introduced using “primer repair”, ligation, in vitro mutagenesis, etc. as appropriate. The vector(s) once completed and demonstrated to have the appropriate sequences may then be introduced into the host cell by any convenient means. The constructs may be integrated and packaged into non-replicating, defective viral genomes like lentivirus, Adenovirus, Adeno-associated virus (AAV), Herpes simplex virus (HSV), or others, including retroviral vectors, for infection or transduction into cells. The vector(s) may include viral sequences for transfection, if desired. Alternatively, the construct may be introduced by fusion, electroporation, biolistics, transfection, lipofection, or the like. The host cells may be grown and expanded in culture before introduction of the vector(s), followed by the appropriate treatment for introduction of the vector(s) and integration of the vector(s). The cells are then expanded and screened by virtue of a marker present in the construct.

1. A method of augmenting efficacy of regenerative cells for repair of ovarian cells and/or tissues, comprising the steps of contacting regenerative cells with one or more biologically active substances; and/or incubating the regenerative cells under conditions to enhance efficacy of said regenerative cells for ovarian repair.

2. The method of claim 1, wherein said cytokine comprises one or more growth factors.

3. The method of claim 2, wherein said growth factor is interleukin-10.

4. The method of claim 2, wherein said growth factor is HGF.

5. The method of claim 2, wherein said growth factor is FGF-beta.

6. The method of claim 2, wherein said growth factor is a member of the TGF-beta family.

7. The method of claim 2, wherein said growth factor is IGF.

8. The method of claim 2, wherein said growth factor is CTGF.

9. The method of claim 1, wherein said biologically active substance comprises platelet rich plasma.

10. The method of claim 1, wherein said regeneration comprises immune modulation.

11. The method of claim 1, wherein the fibroblast cells are selected from the group consisting of (a) regenerative cell obtained by biopsy, cultured and proliferated; and (b) subsets thereof having greater ability to differentiate.

12. The method of claim 1, wherein said regenerative cells are comprised in a pharmaceutically acceptable carrier selected from the group consisting of sterile solutions, hydrogels, implantable cell matrices, devices and a combination thereof.

13. The method of claim 1, wherein the regenerative cells are derived from tissues selected from the group consisting of: skin, heart, blood vessels, bone marrow, skeletal muscle, liver, pancreas, brain, adipose tissue, foreskin, placental, and umbilical cord.

14. The method of claim 1, wherein said biologically active substance is a protease.

15. The method of claim 1, wherein said biologically active substance is a matrix metalloprotease.

16. The method of claim 1, wherein said cell population is allogeneic.

17. The method of claim 1, wherein said cell population is xenogenic.

18. The method of claim 1, wherein said cell population is mesenchymal stem cells.

19. The method of claim 18, wherein said mesenchymal stem cells express IL-1 receptor antagonist when treated with interferon gamma.

20. The method of claim 1, wherein said regenerative cells are administered together with a growth factor.
